(19) United States
(12) Patent Application Publication
(10) Pub. No.: US 2017/0198347 A1
(43) Pub. Date: Jul. 13, 2017

(54) METHODS AND SYSTEMS FOR MEASURING T CELL DIVERSITY

(71) Applicant: THE UNIVERSITY COURT OF THE UNIVERSITY OF GLASGOW, Glasgow (GB)

(72) Inventors: Edward Seungho Lee, Glasgow (GB); Andrew James Yates, Glasgow (GB)

(21) Appl. No.: 15/405,181

(22) Filed: Jan. 12, 2017

(30) Foreign Application Priority Data
Jan. 12, 2016 (GB) 1600584.5

(51) Int. Cl. C12Q 1/68; G06F 19/18; G06F 19/20

(52) U.S. Cl. CPC C12Q 1/6881; G06F 19/20; G06F 19/18; C12Q 2600/158

(57) ABSTRACT

This invention relates to methods and systems for measuring T cell diversity. It is particularly related to methods of identifying T cell receptor (TCR) pairs and their relative frequencies and finds particular use in antigen-specific T cell populations and other T cell populations with limited polyclonality. An exemplary embodiment of the invention provides a method estimating the clonal composition of a T cell population, the method including the steps of: obtaining a plurality of samples from said population, each sample containing a plurality of T cells; sequencing the CDR3α and CDR3β regions of each of those samples; calculating, for every α and β chain found in common in at least one of the samples, an association score which reflects the likelihood of pairing between each of those chains; repeatedly, for a randomly selected subset of said samples, determining, from the calculated association scores, the most likely αβ pairs in each of said selected samples and selecting those pairs as candidate αβ pairs; comparing the number of instances that each of said candidate αβ pairs appears in all of said repeats to a threshold and selecting those pairs whose instances exceed said threshold as true αβ pairs; estimating, from the frequency of co-existence of α and β chains in each of said samples and the sample size of said sample, the frequency of each of said true αβ pairs; and discriminating, amongst said αβ pairs, between possible dual T cell receptors or two clones which share a common β chain.

### REFERENCE TO EARLIER APPLICATIONS

The present application claims priority to GB application no. GB 1600584.5, filed on 12 Jan. 2016, the contents of which are hereby incorporated by reference.

### FIELD OF THE INVENTION

The present invention relates to methods and systems for measuring T cell diversity. It is particularly, but not exclusively, related to methods of identifying T cell receptor (TCR) pairs and their relative frequencies and finds particular use in antigen-specific T cell populations and other T cell populations of limited polyclonality.

### BACKGROUND OF THE INVENTION

The αβ T cell has an essential role in the adaptive immune system. The breadth and specificity of recognition of antigens by T cells is conferred by a process of gene rearrangement that generates a diverse repertoire of T cell receptors (TCR), or clonotypes. The αβ T cell receptor is a heterodimer of an α and a β chain, and each chain contains a hypervariable complementary determining region 3 (CDR3) section that most significantly determines what antigens to which a given TCR can respond. Since the structure of the TCR, particularly the CDR3s, determines its immunogenic properties, T cells can be identified by the genetic sequences encoding the CDR3α and CDRβ regions of their TCRs. There is much interest in determining the CDR3α and CDR3β of clonotypes in the context of infections, autoimmune diseases, and cancer immunology to determine which clonotypes are involved in the immunology of these processes.

One class of methods for approaching this problem uses high throughput sequencing to obtain the CDR3 sequences of one of the two component TCR chains (only TCRα or only TCRβ) used by the T cells in a sample [1], [2]. Simultaneous sequencing of both chains of a large sample of cells causes a problem as data from such a method would yield a set of CDR3α and CDR3β sequences but would not provide information about which sequences pair together from the same TCR.

Another class of methods uses various techniques to separate the T cell sample into single cells and perform high-throughput sequencing of both chains on individual cells, preserving the pairing information [3], [4], [5]. However, such methods are technically challenging, laborious, and unscalable to obtain the paired sequences of thousands of TCRs that can typically be involved in an antigen-specific immune response.

The most recent approaches utilize frequency-based pairing to determine sequence pairs of receptors. The earliest is a rudimentary approach to match the frequencies of sequence reads of individual chains, pairing up sequences back to their receptors based on frequency [6]. A more recently published approach sequences the CDR3α and CDR3β of many large samples of T cells and uses a combinatorics (statistical) approach to resolve heterodimer pairs that originate from a single T cell [7].

However, these methods fail to fully address the complexity of T cell clonality, particularly that within populations of T cells specific for a given epitope (antigen-specific T cells′). First, TCRs from different clonotypes of antigen-specific populations can share a common component chain, as shown in FIG. 1 [3], [4]. Second, a significant proportion of clonotypes can express two different TCRα chains and thus have two TCRs, at least at the sequence level if not both expressed at the cell surface, which appears as one TCRβ chain paired with two different TCRα chains [4]. Finally, the frequencies of clonotypes within antigen-specific populations are skewed, and thus in a pairing strategy based on sampling of cell population, common clones will be sampled more often than rarer clones.

These three unique properties mean that there is a need for a novel computational approach to take high-throughput sequencing data of samples of T cell populations and determine the CDR3α/CDR3β sequences that define each clonotype, particularly for antigen-specific T cell populations.

The combinatorics approach discussed above is used for naive T cells. The process of sequencing and pairing TCR chains of small samples of naive T cell populations, in which individual clonotype frequencies are typically very small, is not expected to encounter sharing of either the α or β chains. As a result the inability of this approach to deal with chain sharing may be a minor issue in such analysis. However sharing has been demonstrated experimentally to be commonly encountered in antigen-specific T cell populations. It is therefore unclear that this existing method can be applied meaningfully to antigen-specific populations.

The present invention aims to provide methods and systems that provide more accurate characterization of T cell populations, in particular antigen-specific T cell populations and other T cell populations of limited polyclonality (i.e. populations that are not as diverse as a population of nave T cells) such as tumor-infiltrating lymphocytes.

### SUMMARY OF THE INVENTION

An exemplary embodiment of the present invention provides a method of estimating the clonal composition of a T cell population, the method including the steps of: taking a plurality of samples from said population, each sample containing a plurality of T cells; sequencing the CDR3α and CDR3β regions of each of those samples; calculating, for every α and β chain found in common in at least one of the samples, an association score which reflects the likelihood of pairing between each of those chains; repeatedly, for a randomly selected subset of said samples, determining, from the calculated association scores, the most likely αβ pairs in each of said samples and selecting those pairs as candidate αβ pairs; comparing the number of instances that each of said candidate αβ pairs appears in each of said repeats to a threshold and selecting those pairs whose instances exceed said threshold as true αβ pairs; estimating, from the frequency of co-existence of α and β chains in each of all samples and the sample size of said sample, the frequency of each of said true αβ pairs; and discriminating, amongst said αβ pairs, between possible dual T cell receptor clones or two clones which share a common β chain.

A further exemplary embodiment of the present invention provides a method of estimating the clonal composition of a T cell population, the method including the steps of: taking a plurality of samples from said population, each sample containing a plurality of T cells; sequencing the CDR3α and CDR3β regions of each of those samples; repeatedly, for a randomly selected subset of said samples: calculating, for every α and β chain found in common in at least one of the samples in the subset, an association score which reflects the likelihood of pairing between each of those chains; and determining, from the calculated association scores, the most likely αβ pairs in each of said selected samples and selecting those pairs as candidate αβ pairs; comparing the number of instances that each of said candidate αβ pairs appears in all of said repeats to a threshold and selecting those pairs whose instances exceed said threshold as true αβ pairs; estimating, from the frequency of co-existence of α and β chains in each of said samples and the sample size of said sample, the frequency of each of said true αβ pairs; and discriminating, amongst said αβ pairs, between possible dual T cell receptors or two clones which share a common β chain.

A further exemplary embodiment of the present invention provides a system for estimating the clonal composition of a T cell population, the system having: a plurality of sample holders each for receiving a plurality of samples from said population, each sample containing a plurality of T cells; a sequencer for sequencing the samples; and a processor, wherein: the sequencer sequences the CDR3α and CDR3β regions of each of the samples; the processor is arranged to: calculate, for every α and β chain found in common in each of said selected samples, an association score which reflects the likelihood of pairing between each of those chains; repeatedly, for a randomly selected subset of said samples: determine, from the calculated association scores, the most likely αβ pairs in each of said samples and selecting those pairs as candidate αβ pairs; compare the number of instances that each of said candidate αβ pairs appears in all of said repeats to a threshold and selecting those pairs whose instances exceed said threshold as true αβ pairs; estimate, from the frequency of co-existence of α and β chains in each of all samples and the sample size of said sample, the frequency of each of said true αβ pairs; and discriminate, amongst said αβ pairs, between possible dual T cell receptors or two clones which share a common β chain.

A further exemplary embodiment of the present invention provides a system for estimating the clonal composition of a T cell population, the system having: a plurality of sample holders each for receiving a plurality of samples from said population, each sample containing a plurality of T cells; a sequencer for sequencing the samples; and a processor, wherein: the sequencer sequences the CDR3α and CDR3β regions of each of the samples; the processor is arranged to: repeatedly, for a randomly selected subset of said samples: calculate, for every α and β chain found in common in at least one of the samples in the subset, an association score which reflects the likelihood of pairing between each of those chains; and determine, from the calculated association scores, the most likely αβ pairs in each of said samples and selecting those pairs as candidate αβ pairs; compare the number of instances that each of said candidate αβ pairs appears in all of said repeats to a threshold and selecting those pairs whose instances exceed said threshold as true αβ pairs; estimate, from the frequency of co-existence of α and β chains in each of said samples and the sample sizes, the frequency of each of said true αβ pairs; and discriminate, amongst said αβ pairs, between possible dual T cell receptors or two clones which share a common β chain.

### BRIEF DESCRIPTION OF THE DRAWINGS

Embodiments of the invention will now be described by way of example with reference to the accompanying drawings in which:

FIG. 1 shows how antigen-specific T cell populations may contain clones that share common α or β and/or dual TCR clones that express two α chains;

FIG. 2 is a flow chart setting out the basic steps in a method according to an embodiment of the present invention;

FIG. 3 shows an initial step of sorting samples into wells of 96-well plates;

FIG. 4 shows the results of a random selection of 75% of the wells on a well plate;

FIG. 5 shows the calculation of scores to determine the most likely αβ pairs that make up the TCRs from the clones sampled in a well;

FIG. 6 shows the selection of candidate pairs from the selected pairs;

FIG. 7 shows the application of a maximum likelihood approach to estimate the frequency of each clone;

FIG. 8 illustrates the results of the clustering step in a method according to an embodiment of the present invention;

FIG. 9 shows the overall depth of the population in results from a simulation of a method according to an embodiment of the present invention;

FIG. 10 shows the false positive rate in results from a simulation of a method according to an embodiment of the present invention;

FIG. 11 shows the depth of top clones in results from a simulation of a method according to an embodiment of the present invention;

FIG. 12 shows the adjusted depth of dual clones in results from a simulation of a method according to an embodiment of the present invention;

FIG. 13 shows the false dual clone rate in results from a simulation of a method according to an embodiment of the present invention;

FIG. 14 shows the top dual depth in results from a simulation according to an embodiment of the present invention;

FIG. 15 shows the associated false dual clone rate from the same simulation as FIG. 14;

FIG. 16 shows a comparison of the performance between a method according to an embodiment of the present invention and single-cell sequencing performed on identical simulations of T cell populations;

FIG. 17 shows similar simulations to those compared in FIG. 16 but with different skewnesses;

FIG. 18 shows a comparison of the TCR clonotypes predicted from a published dataset by a prior art method compared to methods according to embodiments of the present invention; and

FIG. 19 shows the same comparison as FIG. 18 but broken down between the individual tumors in the published dataset.

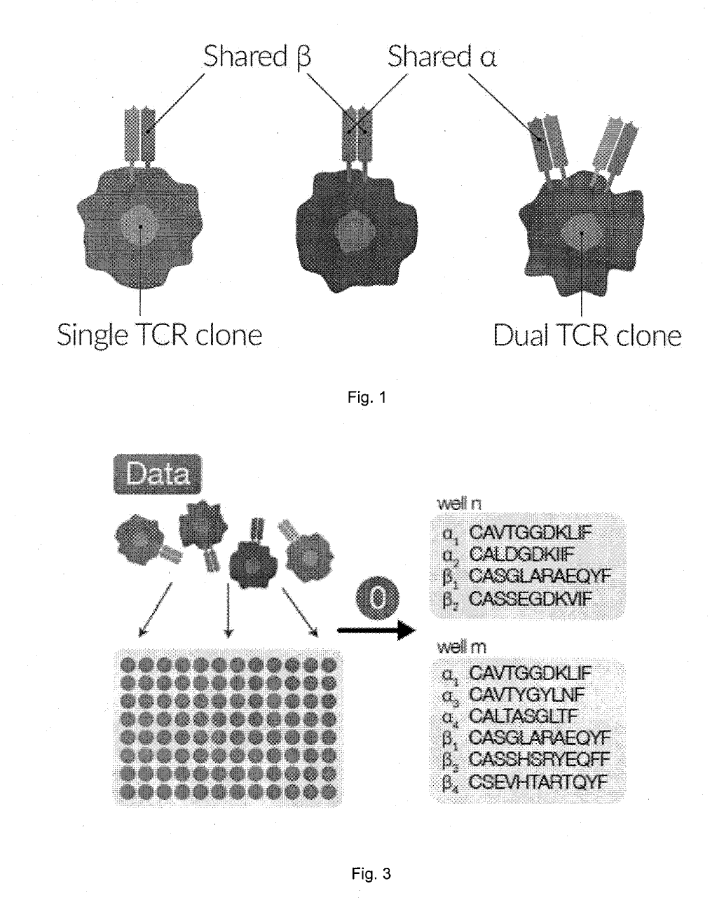

### DETAILED DESCRIPTION

At their broadest, aspects of the present invention provide methods and systems for estimating the clonal composition of a T cell population by determining association scores between α and β chains that are found together in samples of the population and repeatedly using those scores to determine the most likely αβ pairs.

A first aspect of the present invention provides a method of estimating the clonal composition of a T cell population, the method including the steps of: taking a plurality of samples from said population, each sample containing a plurality of T cells; sequencing the CDR3α and CDR3β regions of each of those samples; calculating, for every α and β chain found in common in at least one of the samples, an association score which reflects the likelihood of pairing between each of those chains; repeatedly, for a randomly selected subset of said samples, determining, from the calculated association scores, the most likely αβ pairs in each of said samples and selecting those pairs as candidate αβ pairs; comparing the number of instances that each of said candidate αβ pairs appears in each of said repeats to a threshold and selecting those pairs whose instances exceed said threshold as true αβ pairs; estimating, from the frequency of co-existence of α and β chains in each of all samples and the sample size of said sample, the frequency of each of said true αβ pairs; and discriminating, amongst said αβ pairs, between possible dual T cell receptor clones or two clones which share a common β chain.

A second aspect of the present invention provides a method of estimating the clonal composition of a T cell population, the method including the steps of: taking a plurality of samples from said population, each sample containing a plurality of T cells; sequencing the CDR3α and CDR3β regions of each of those samples; repeatedly, for a randomly selected subset of said samples: calculating, for every α and β chain found in common in at least one of the samples in the subset, an association score which reflects the likelihood of pairing between each of those chains; and determining, from the calculated association scores, the most likely αβ pairs in each of said selected samples and selecting those pairs as candidate αβ pairs; comparing the number of instances that each of said candidate αβ pairs appears in all of said repeats to a threshold and selecting those pairs whose instances exceed said threshold as true αβ pairs; estimating, from the frequency of co-existence of α and β chains in each of said samples and the sample size of said sample, the frequency of each of said true αβ pairs; and discriminating, amongst said αβ pairs, between possible dual T cell receptors or two clones which share a common β chain.

The methods of the above aspects have been shown to be successful in providing a good proportion of αβ pairs which are correctly identified and a reduced proportion of αβ pairs which are incorrectly identified (false positives). By increasing the number of samples, the success rates can be improved further with less false positives.

The methods of these aspects also require no assumptions regarding the number of different β chains that an α chain can pair with, and vice-versa. This freedom makes it possible to pair shared chains.

By performing the frequency estimation, the discriminating step becomes possible and allows discrimination between possible dual T cell receptor clones or two clones which share a common β chain.

The following optional and preferred features apply to both of the above aspects.

In certain embodiments the T cell population is antigen-specific. Antigen-specific means that the population of T cells is specific for a given epitope. Herein, the term antigen-specific will be used to refer to T cell populations in which at least 90%, preferably 95% and more preferably 99% of the cells are specific for a single epitope. That is to say, the respective cells are specific for the same single epitope. The method of the present aspect has been shown to work particularly well on such T cell populations and addresses the specific issues associated with determining pairing in antigen-specific T cell populations as indicated above.

In other embodiments the T cell population has limited clonal diversity (e.g. tumor-infiltrating lymphocytes (TILs) or T cell populations involved in an autoimmune disease). TILs are T cells found within a tumor and are presumed to be trying to respond to the tumor. They may or may not be responding to the same single epitopes.

Preferably the association score for a particular combination of α and β chains is calculated as a function that increases with the number of concurrent appearances of said combination in the samples, weighted inversely by the number of unique α and β chains found in each of the samples in which the combination is present. This allows the likelihood of association that arises from finding a particular combination of α and β chains in a group to be weighted based on the probability that those α and β chains form pairs with other chains in that sample.

Preferably the random selection is of between 60% and 90% of said samples, more preferably between 70% and 80% of said samples and most preferably approximately or exactly 75% of said samples.

Preferably the repeated steps in the method are performed at least 100 times. The number of repeats ensures that there is sufficient data for the statistical effects of the method to work.

The step of determining the likely αβ pairs may involve using a linear sum assignment problem. Preferably such linear sum assignment problem is solved using an algorithm such as the Hungarian algorithm.

The threshold can be defined in terms of the proportion of repeats in which a candidate αβ pair appears. This can be selected by the user, depending on the desired results. A lower threshold (e.g. at least 60%) will tend to increase the number of αβ pairs that are correctly identified, but also increase the number of false positives. A higher threshold (e.g. at least 90%) tends to decrease the number of correct pairs that are identified but also lowers the number of false positives. Thus the threshold can be set by the user depending on if they want to identify more pairs or minimize the false positives. Intermediate thresholds (e.g. 70%, 75% or 80%) can also be selected.

Preferably the step of estimating the frequency uses a maximum likelihood approach.

Preferably the step of discriminating includes determining the ratio of the determined number of co-occurrences of the possible clones in the groups to the number expected if all pairs were distinct clones. More preferably this step further uses k-means clustering to partition the pairings into a group of dual clones and a group of β-sharing clones.

Optionally the step of discriminating may include, or further include, determining the likelihoods of all occurrences of dual clones and β-sharing clones under the alternative hypotheses of the clones being dual clones or being β-sharing clones and comparing the differences in the likelihoods obtained.

In preferred embodiment each group is a well in a well plate. For example the samples may be sorted into the wells of standard 96-well plates.

Preferably each sample contains between 20 and 500 T cells, more preferably between 20 and 300 T cells.

In certain embodiments of the present aspect, a plurality of said samples have a different number of T cells in their sample to each other. For example, some samples may have 20 T cells, some 50, some 100, some 200 and some 300, or any combination of two or more of these. Such mixed sampling strategies have been found to be optimal for moderately skewed clonal distributions in reducing false positive rates and increasing the percentage of the most abundant clones which are correctly identified.

The methods of these aspects may include any combination of some, all or none of the above described preferred and optional features.

The methods of the above first and second aspects are preferably implemented by systems according to the third and fourth aspects of this invention respectively, as described below, but need not be.

Further aspects of the present invention include computer software which, when run on a computer, carry out the steps of any of the above methods, including some, all or none of the preferred or optional features of those methods.

A third aspect of the present invention provides a system for estimating the clonal composition of a T cell population, the system having: a plurality of sample holders each for receiving a plurality of samples from said population, each sample containing a plurality of T cells; a sequencer for sequencing the samples; and a processor, wherein: the sequencer sequences the CDR3α and CDR3β regions of each of the samples; the processor is arranged to: calculate, for every α and β chain found in common in each of said selected samples, an association score which reflects the likelihood of pairing between each of those chains; repeatedly, for a randomly selected subset of said samples: determine, from the calculated association scores, the most likely αβ pairs in each of said samples and selecting those pairs as candidate αβ pairs; compare the number of instances that each of said candidate αβ pairs appears in all of said repeats to a threshold and selecting those pairs whose instances exceed said threshold as true αβ pairs; estimate, from the frequency of co-existence of α and β chains in each of all samples and the sample size of said sample, the frequency of each of said true αβ pairs; and discriminate, amongst said αβ pairs, between possible dual T cell receptors or two clones which share a common β chain.

A fourth aspect of the present invention provides a system for estimating the clonal composition of a T cell population, the system having: a plurality of sample holders each for receiving a plurality of samples from said population, each sample containing a plurality of T cells; a sequencer for sequencing the samples; and a processor, wherein: the sequencer sequences the CDR3α and CDR3β regions of each of the samples; the processor is arranged to: repeatedly, for a randomly selected subset of said samples: calculate, for every α and β chain found in common in at least one of the samples in the subset, an association score which reflects the likelihood of pairing between each of those chains; and determine, from the calculated association scores, the most likely αβ pairs in each of said samples and selecting those pairs as candidate αβ pairs; compare the number of instances that each of said candidate αβ pairs appears in all of said repeats to a threshold and selecting those pairs whose instances exceed said threshold as true αβ pairs; estimate, from the frequency of co-existence of α and β chains in each of said samples and the sample sizes, the frequency of each of said true αβ pairs; and discriminate, amongst said αβ pairs, between possible dual T cell receptors or two clones which share a common β chain.

The systems of these aspects have been shown to be successful in providing a good proportion of αβ pairs which are correctly identified and a reduced proportion of αβ pairs which are incorrectly identified (false positives). By increasing the number of samples, the success rates can be improved further.

The systems of these aspects require no assumptions regarding the number of different β chains that an α chain can pair with, and vice-versa. This freedom makes it possible to pair shared chains.

By performing the frequency estimation, the final discrimination becomes possible and allows the processor to discriminate between possible dual T cell receptor clones or two clones which share a common β chain.

The following optional and preferred features apply to both of the above third and fourth aspects.

In certain embodiments the T cell population is antigen-specific. Antigen-specific means that the population of T cells is specific for a given epitope. Herein, the term antigen-specific will be used to refer to T cell populations in which at least 90%, preferably 95% and more preferably 99% of the cells are specific for a single epitope. That is to say, the respective cells are specific for the same single epitope. The system of the present aspect has been shown to work particularly well on such T cell populations and addresses the specific issues associated with determining pairing in antigen-specific T cell populations as indicated above.

In other embodiments the T cell population has limited clonal diversity (e.g. tumor-infiltrating lymphocytes (TILs) or T cell populations involved in an autoimmune disease). TILs are T cells found within a tumor and are presumed to be trying to respond to the tumor. They may or may not be responding to the same single epitopes.

Preferably the association score for a particular combination of α and β chains is calculated as a function that increases with the number of concurrent appearances of said combination in the groups, weighted inversely by the number of unique α and β chains found in each of the samples in which the combination is present. This allows the likelihood of association that arises from finding a particular combination of α and β chains in a group to be weighted based on the probability that those α and β chains form pairs with other chains in that sample.

Preferably the random selection is of between 60% and 90% of said samples, more preferably between 70% and 80% of said samples and most preferably approximately or exactly 75% of said samples.

Preferably the processor performs the repeated steps at least 100 times. The number of repeats ensures that there is sufficient data for the statistical effects of the method to work.

The step of determining the likely αβ pairs may involve using a linear sum assignment problem. Preferably such linear sum assignment problem is solved using an algorithm such as the Hungarian algorithm.

The threshold can be defined in terms of the proportion of repeats in which a candidate αβ pair appears. This can be selected by the user, depending on the desired results. A lower threshold (e.g. at least 60%) will tend to increase the number of αβ pairs that are correctly identified, but also increase the number of false positives. A higher threshold (e.g. at least 90%) tends to decrease the number of correct pairs that are identified but also lowers the number of false positives. Thus the threshold can be set by the user depending on if they want to identify more pairs or minimize the false positives. Intermediate thresholds (e.g. 70%, 75% or 80%) can also be selected.

Preferably the processor estimates the frequencies using a maximum likelihood approach.

Preferably the processor is arranged to perform said discrimination by determining the ratio of the determined number of co-occurrences of the possible clones in the samples to the number expected if all pairs were distinct clones. More preferably the processor further uses k-means clustering to partition the pairings into a group of dual clones and a group of β-sharing clones.

Optionally the processor is, alternatively or additionally, arranged to perform said discrimination by determining the likelihoods of all occurrences of dual clones and β-sharing clones under the alternative hypotheses of the clones being dual clones or being β-sharing clones and comparing the differences in the likelihoods obtained.

In preferred embodiment each sample holder is a well in a well plate. For example the samples may be sorted into the wells of standard 96-well plates.

Preferably each sample contains between 20 and 500 T cells, more preferably between 20 and 300 T cells.

In certain embodiments of the present aspect, a plurality of said samples have a different number of T cells to each other. For example, some samples may have 20 T cells, some 50, some 100, some 200 and some 300 or any combination of two or more of these. Such mixed sampling strategies have been found to be optimal for moderately skewed clonal distributions in reducing false positive rates and increasing the percentage of the most abundant clones which are correctly identified.

The systems of the present aspects may include any combination of some, all or none of the above described preferred and optional features.

The systems of the present aspect may operate by carrying out a method according to the first or second aspects of this invention described above, but need not do so.

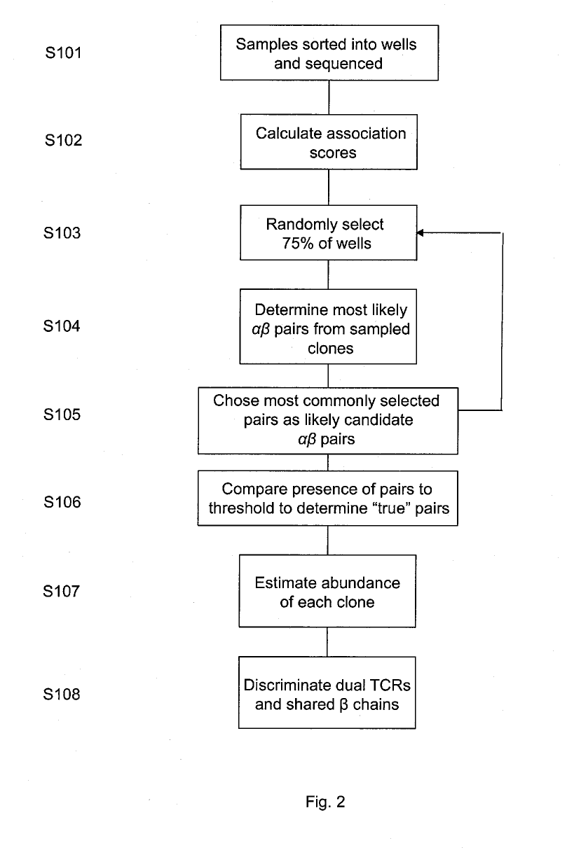

An embodiment of a method according to the present invention is illustrated in the flowchart of FIG. 2. FIGS. 3-9 further illustrate some of the steps of this method.

The method is implemented as a computer algorithm which determines CDR3α/CDR3β pairs from sequencing data from multiple samples of 20-300 T cells (96-480 samples) from antigen-specific populations and other populations of limited polyclonality such as tumour-infiltrating lymphocytes.

These samples are placed in the wells of 96-well plates, and each well represents a sample of 20-300 T cells. The samples are sequenced (S101). Each sample yields a readout of the unique CDR3α and CDR3β sequences of the clonotypes sampled in that well. An example sequencing method which yields the CDR3α and CDR3β sequences from T cells is given in [7] and is hereby incorporated by reference.

The data yields a set of all of the unique α and β chains found in all of the wells and in which wells each chain was found. For the steps below, the following parameters are defined: the data contains Wwells, g unique α chains, and h unique β chains.

Step 1: The algorithm begins by calculating an association scores between every α and β chain (S102). The association between chains is measured by a score Sᵢⱼ for chains αᵢ and βⱼ which is calculated as

$${S}_{ij} = {\sum }_{k = 1}^{W} ( \frac{{δ}_{ij}^{( k )}}{{c}_{α}^{( k )}} \cdot \frac{{δ}_{ij}^{( k )}}{{c}_{β}^{( k )}} ) \qquad ( Equation 1 )$$

where the wells are labelled from 1, 2, . . . , W, the number of distinct α and β chains in well k are c_{α}^{(k)} and c_{β}^{(k)} respectively, and δᵢⱼ^{(k)} is 1 if both αᵢ and βⱼ are found in well k and 0 otherwise. Equation (1) is constructed to add up the number of concurrent well appearances while inversely scaling each concurrent appearance by the number of partner chains in the well.

There is flexibility in the definition of the association score (Equation 1). It is an empirically-determined measure of support for pairing, based on the number of times the chains appear in a well together, with each co-incidence inversely weighted by the total number of unique α or β chains recovered from that well (the fewer chains that are in a well, the greater the confidence that two chains are associated).

Variations in this function are possible. Examples include

$${S}_{ij} = {\sum }_{k = 1}^{W} ( \frac{{δ}_{ij}^{( k )}}{{c}_{α}^{{( k )}^{2}}} + \frac{{δ}_{ij}^{( k )}}{{c}_{β}^{{( k )}^{2}}} ) and {S}_{ij} = {\sum }_{k = 1}^{W} ( \frac{{δ}_{ij}^{( k )}}{{c}_{α}^{( k )}} \cdot \frac{{δ}_{ij}^{( k )}}{{c}_{β}^{( k )}} )$$

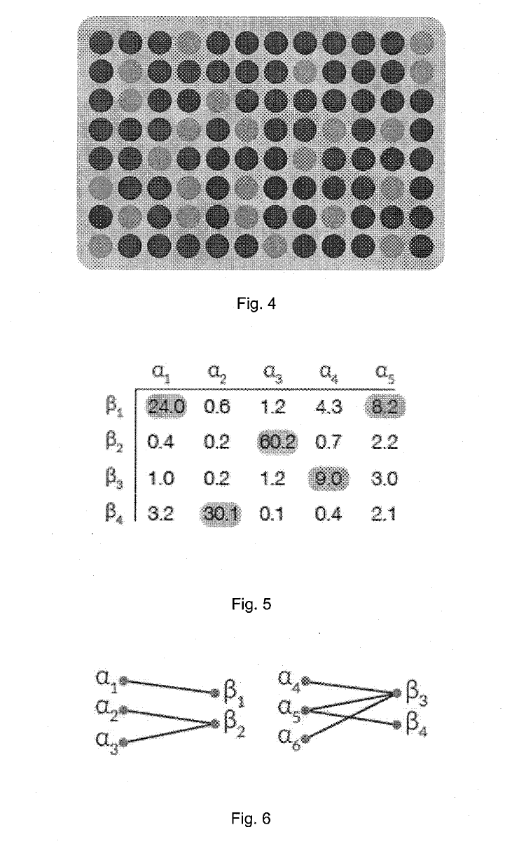

Step 2: The algorithm takes a subset of the data set by choosing randomly 75% of the wells (S103) and then performs Steps 3 to 4 below. This selection is illustrated in FIG. 4. The subsequent steps are repeated a plurality of times as decided by the user. The number of repetitions is ideally at least 100 to ensure sufficient statistical power for the further steps.

The selection of a random subset of the data set is important as this enables the statistical methods that follow to lower the false positive rate in finding matches between α and β chains. The selection of 75% of the wells was determined heuristically to be optimal, but other selections are possible, for example between 60-90%, or 70-80%.

In an alternative embodiment of the present invention, Step 1 described above can be performed after the selection of the subset, and performed repeatedly for each subset selected.

Step 3: The algorithm then loops through each well and decides which pairs of the α and β chains found in the well are most likely to originate from the same TCR (S104). Aₖ is defined to denotes the set of A distinct α chains found in well k, that is Aₖ={αₘ_{₁}_{^{k}}, αₘ_{₂}_{^{k}}, . . . , αₘ_{_{A}}_{^{k}}} , where the mᵢ^{k} ∈ {1, . . . , N_{α}}, are integers that denote the labels of the A TCRα chains found in well k. Similarly Bₖ is defined to denote the set of B distinct β chains found in well k, that is Bₖ={βₙ_{₁}_{^{k}}, βₙ_{₂}_{^{k}}, . . . , βₙ_{_{B}}_{^{k}}} where the nᵢ^{k} ∈ {1, . . . , N_{β}} subscripts denote the labels of the B TCRβ chains found in well k. The method solves the following linear sum assignment problem using the Hungarian algorithm (other algorithms and other formulations of the linear sum assignment problem may also used for this step):

$$maximize \\ {\sum }_{{α}_{i} ∈ {𝒜}_{k}} {\sum }_{{β}_{i} ∈ {ℬ}_{k}} {S}_{ij} {x}_{ij} \\ subject to \\ {\sum }_{{α}_{i} ∈ {𝒜}_{k}} {x}_{ij} = 1 for {β}_{j} ∈ {ℬ}_{k} \\ {\sum }_{{β}_{i} ∈ {ℬ}_{k}} {x}_{ij} = 1 for {α}_{i} ∈ {𝒜}_{k} \\ {x}_{ij} \geq 0 , {α}_{i} ∈ {ℬ}_{k} , {β}_{j} ∈ {𝒜}_{k} \qquad ( Equation 2 )$$

where xᵢⱼ=1 indicates αᵢ and βⱼ are assigned as a candidate TCR pair and xᵢⱼ=0 otherwise. A pair αᵢ and βⱼ is defined as an assigned pair of well k if xᵢⱼ=1 for Equation (2) associated with well k. The number of assignments made for every pair of α and β is recorded as Xᵢⱼ, i.e. Xᵢⱼ equals the number of times xᵢⱼ=1 arises from the solutions of Equation (2) for each well. The association scores and the identified pairs are illustrated in FIG. 5.

Step 4: A threshold T is calculated as the mean of the elements of the set {N (i, j): N (i, j) >0, i ∈ 1,2, . . . , g, j ∈ 1,2, . . . , h}, where N (i, j) is the number of times αᵢβⱼ are assigned to each other. The output of this algorithm then is a list of αᵢ and βⱼ pairs such that Xᵢⱼ>T chain pairs that form TCR pairs (S105). The threshold can be calculated in other ways too, such as from the median of the elements of the same set, or by taking the mean±one standard deviation.

Step 5: Each replicate of Steps 2-4 yields a list of candidate αᵢβⱼ pairs. The pairs that appear in more than a proportion p_{R} of replicates are chosen as true TCR pairs (S106). The threshold can be selected by the user, depending on the desired results. A lower threshold (e.g. at least 60%) will tend to increase the number of αβ pairs that are correctly identified, but will also tend to increase the number of false positives. A higher threshold (e.g. at least 90%) tends to decrease the number of correct pairs that are identified but also tends to lower the number of false positives. An example selection of true TCR pairs is shown in FIG. 6.

Step 6: The algorithm then uses a maximum likelihood approach to estimate the frequency of each clone identified in Step 5 (S107). Let P be the number of 96-well plates in the data, K={k₁, k₂, . . . , k₁₂} be a vector where element kⱼ represents the number of cells in each of the wells of column j, cᵢⱼ be the clone with chains αᵢ and βⱼ, and wᵢⱼ^{l} be the number of wells of column/in which clone cᵢⱼ appears. Clone cᵢⱼ is defined to appear in a well if αᵢ and βⱼ are found in that well. Then, for each clone, the likelihood that clone cᵢⱼ has a relative frequency fᵢⱼ ∈ (0; 1] is

$$\begin{matrix}ℒ ( clone {c}_{ij} has frequency {f}_{ij} ) = {\prod }_{l = 1}^{12} ( \begin{matrix}8 P \\ {w}_{ij}^{l}\end{matrix} ) {( 1 - {q}_{l} )}^{{w}_{ij}^{l}} {{q}_{l}}^{8 P - {w}_{ij}^{l}} \\ where & ( Equation 3 ) \\ {q}_{l} = {( 1 - {f}_{ij} )}^{{k}_{l}} + {\sum }_{m = 1}^{{k}_{l}} ( 2 {ε}^{m} - {ε}^{2 m} ) ( \begin{matrix}{k}_{l} \\ m\end{matrix} ) {{f}_{ij}^{l} ( 1 - {f}_{ij} )}^{{k}_{l} - m} & ( Equation 4 )\end{matrix}$$

where ∈ is the experimental error ‘drop’ rate, which may be the average probability that a CDR3 sequence in a cell fails to be amplified and sequenced.

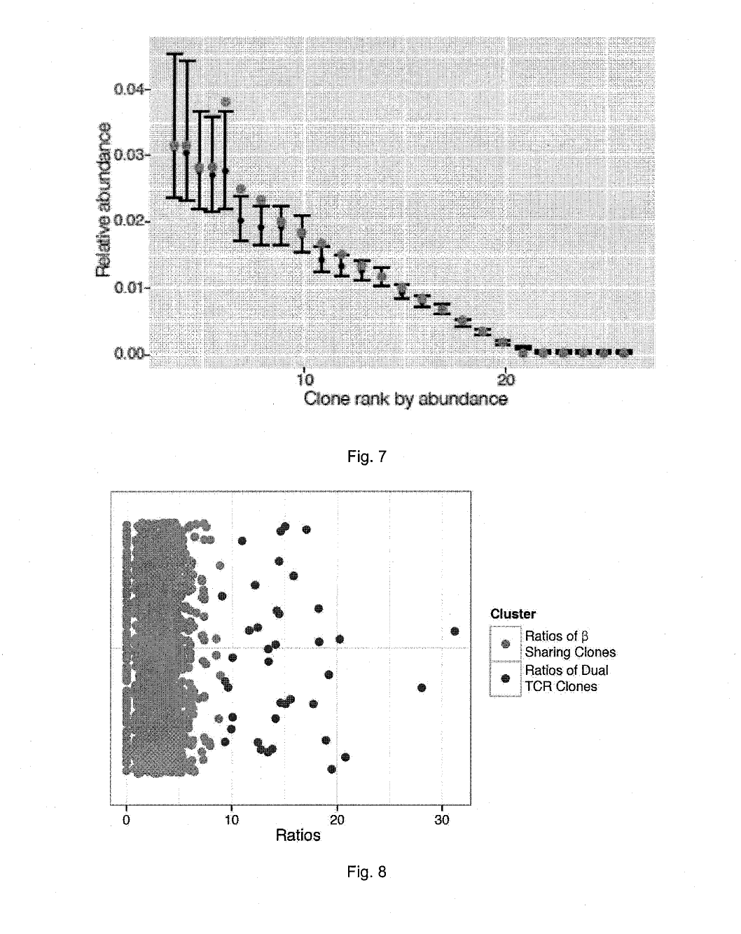

For every clone cᵢⱼ the algorithm maximizes Equation (3) to estimate its frequency fᵢⱼ, and 95% confidence intervals are calculated by estimating parameter values yielding log L=log Lₘₐₓ−1.96. The results from a simulated data set are shown in FIG. 7.

The maximum likelihood function can be modified according to the specifics of the dataset (in the above function, it is assumed that the samples are contained in a 96-well plate set up as 8 rows by 12 columns, and that the wells of each column have the same sample size; obviously the function can be modified for other well plates and to allow for arbitrary sample sizes in each well). Further, it may be possible to remove the summation term from Equation (4) if the experimental error rate E is sufficiently low.

For example, a generalised form of the above equations may be derived by letting N={n₁, n₂, . . . , nₛ} be the set of s distinct sample sizes (nᵢ cells per well) in all of the wells and W={w₁, w₂, . . . , wₛ} where wᵢ represents the number of wells with samples of size nᵢ cells. Let cᵢⱼ denote the clone with chains αᵢ and βⱼ and kᵢⱼ^{l} denote the number of wells of sample size nₗ cells per well that contain chains αᵢ and βⱼ. The likelihood of the observations kᵢⱼ^{(.)}, given that the clone cᵢⱼ is present at frequency fᵢⱼ within the population, is

$$ℒ ( observed incidence of clone {c}_{ij} \vert {f}_{ij} ) = {\prod }_{l = 1}^{s} ( \begin{matrix}{w}_{l} \\ {k}_{ij}^{l}\end{matrix} ) {( 1 - {q}_{l} )}^{{k}_{ij}^{l}} {q}_{l}^{{w}_{l} - {k}_{ij}^{l}} \qquad ( Equation 3 A )$$

where qₗ is the probability of clone cᵢⱼ not being found in well/and is given by

$${q}_{l} = {( 1 - {f}_{ij} )}^{{n}_{l}} + {\sum }_{m = 1}^{{n}_{l}} ( 2 {ε}^{m} - {ε}^{2 m} ) ( \begin{matrix}{n}_{l} \\ m\end{matrix} ) {{f}_{ij}^{l} ( 1 - {f}_{ij} )}^{{n}_{l} - m} \qquad ( Equation 4 A )$$

The step then proceeds by maximizing Equation (3A) to estimate its frequency fᵢⱼ, and 95% confidence intervals are calculated by estimating parameter values yielding log L=log Lₘₐₓ−1.96.

This procedure is applied to every αβ pair identified in the first phase of the algorithm. These estimated frequencies can be used to distinguish TCRβ-sharing clone pairs from single TCR clones expressing two TCRα. This procedure is described in the following optional Step 7a.

When a clone with two TCRα is identified, the frequency estimate is revised as follows. Let c₍ᵢⱼ₎ₜ denote a clone with chains αᵢ, αⱼ and βₜ and k₍ᵢⱼ₎ₜ^{l} denote the number of wells of size nₗ that contain chains αᵢ, aⱼ and βₜ. The likelihood of the observations given that clone c₍ᵢⱼ₎ₜ has a frequency f_{(i,j)t} ∈ (0,1] is

$$ℒ ( observed incidence of clone {c}_{( ij ) t} \vert {f}_{( ij ) t} ) = {\prod }_{l = 1}^{s} ( \begin{matrix}{w}_{l} \\ {k}_{( ij ) t}^{l}\end{matrix} ) {( 1 - {q}_{l} )}^{{k}_{( ij ) t}^{l}} {q}_{l}^{{w}_{l} - {k}_{( ij ) t}^{l}} \qquad ( Equation 3 B )$$

where qₗ is the probability of clone c₍ᵢⱼ₎ₜ not being found in well/and is given by

$${q}_{l} = {( 1 - {f}_{ij} )}^{{n}_{l}} + {\sum }_{m = 1}^{{n}_{l}} ( 3 {ε}^{m} - 3 {ε}^{2 m} + {ε}^{3 m} ) ( \begin{matrix}n \\ m\end{matrix} ) {{f}_{( ij ) t}^{m} ( 1 - {f}_{( ij ) t} )}^{{n}_{l} - m} \qquad ( Equation 4 A )$$

where ∈ is the mean drop rate as described above. Equation 3B is then maximised to estimate f₍ᵢⱼ₎ₖ, and again log L=log Lₘₐₓ−1.96 is used to calculate 95% confidence intervals.

Step 7: The algorithm then discriminates between dual TCRs and shared beta chains (S108). Using the frequency estimates in Step 6, the algorithm calculates the following set of values

$$R = \{ {r}_{ik}^{j} = \frac{A ( {c}_{ij} , {c}_{kj} )}{E ( {c}_{ij} , {c}_{kj} )} \text{:} i \neq k , j ∈ 1 , 2 , … , {N}_{β} \} \qquad ( Equation 5 )$$

where A(cᵢⱼ,cₖⱼ) is the number of times clones cᵢⱼ and cₖⱼ appear in the same well in the data, and N_{β} is the number of distinct β chains, and the expected number is:

$$E ( {c}_{ij} , {c}_{kj} ) = {\sum }_{l = 1}^{12} 8 P ( 1 - {( 1 - {f}_{ij} )}^{{w}_{ij}^{l}} - {( 1 - {f}_{kj} )}^{{w}_{ij}^{l}} + {( 1 - {f}_{ij} - {f}_{kj} )}^{{w}_{ij}^{l}} ) \qquad ( Equation 6 )$$

which may be generalised for well-plates of all dimensions (using the notations from Step 6 above) as:

$$E ( {c}_{ij} , {c}_{kj} ) = {\sum }_{l = 1}^{s} {w}_{l} ( 1 - {( 1 - {f}_{ij} )}^{{n}_{l}} - {( 1 - {f}_{kj} )}^{{n}_{l}} + {( 1 - {f}_{ij} - {f}_{kj} )}^{{n}_{l}} ) \qquad ( Equation 6 A )$$

The ratios in set R are partitioned into two groups C₁ and C₂ using k-means clustering, where the mean of ratios of C₁ is greater than the mean of the ratios of C₂. The clones associated with the ratios in C₁ are chosen as dual TCR clones, such that if rᵢₖ^{j} ∈ C₁, then clones cᵢⱼ and cₖⱼ are removed from the list of TCR pairs and replaced with a dual clone made up of chains αᵢαₖβⱼ. FIG. 8 shows the results of this clustering exercise.

Step 7a: However, performing k-means clustering on only the numbers of three-way occurrences can be inefficient at discriminating β-sharing and dual TCRα clones that are relatively abundant because the expected frequencies of co-occurrences become indistinguishable, particularly for rich sampling strategies in which the three chains will co-occur in many wells. In such situations a further step may be used to distinguish between these possibilities. In this, the likelihoods of observed co-occurrences of the three chains are used to assess the relative support for the two alternatives.

In general terms if cᵢⱼ=(αᵢ, βⱼ) and cₖⱼ=(αₖ, βⱼ) are two putative clones with a common beta chain βⱼ, the number of wells containing all three-way, two-way, and single appearances of the three chains are counted. The ‘full’ likelihoods of this pattern of occurrences are then calculated under two hypotheses: (A) that cᵢⱼ and cₖⱼ are indeed two β-sharing clones, with frequencies fᵢⱼ and fₖⱼ estimated using Equation 3 or 3A above; and (B) that the chains derive from one dual TCRα clone c₍ᵢⱼ₎ₖ present at frequency f₍ᵢⱼ₎ₖ, estimated using Equation 3B above. If the difference log L_{A}−log L_{B}≧10, it is assumed the three chains derive from dual TCRα clone.

In more detail, discriminating between relatively abundant β-sharing clones and dual TCRα clones requires comparing the likelihoods of the data under these two hypotheses. Let α_{q}β and αᵣβ be a pair of candidate TCRs that share the same β chain with frequencies f_{q} and fᵣ respectively. The aim is to determine if these two candidate TCRs derive from two β-sharing clones with chains α_{q}β and αᵣβ or from one dual TCRα clone with chains α_{q}αᵣβ. Given the data have wells of s distinct sample sizes n₁, n₂, . . . , nₛ (cells per well), the numbers of wells of each sample size that contain all three chains (α_{q}, αᵣ, β) or contain only two of the three are recorded.

For i ∈ {1, 2, . . . , s}, let
kᵢ¹=the number of wells of sample size nᵢ containing chains β and α_{q} only
kᵢ²=the number of wells of sample size nᵢ containing chains β and αᵣ only
kᵢ³=the number of wells of sample size nᵢ containing chains α_{q} and αᵣ only
kᵢ^{d}=the number of wells of sample size nᵢ containing chains β, α_{q}, and αᵣ
kᵢ^{o}=wᵢ−kᵢ¹−kᵢ²−kᵢ³−kᵢ^{d} be the number of wells of sample size nᵢ that contain none of the chains or exactly one of the three chains

For chains a, b, and c, let W_{abc}ⁱ, W_{ab}ⁱ, Wₐⁱ , and W_{ø}ⁱ denote the events of finding exactly chains a, b, and c, finding exactly chains a and b, finding exactly chain a, and finding none of the chains in a well of sample size nᵢ respectively. Let wᵢ denote the number of wells with sample size nᵢ cells per well, and let s be the number of distinct sample sizes in the data.

The likelihood of observing the data K={kᵢ¹, kᵢ², kᵢ³, kᵢ^{d}, kᵢ^{o}: k=1, 2, . . . , s} under the hypothesis that α_{q}β and αᵣβ represent two β-sharing clones is

$$ℒ ( 𝒦 \vert clone {α}_{q} β with frequency {f}_{q} , clone {α}_{r} β with frequency {f}_{r} ) = {\prod }_{i = 1}^{s} ( \frac{{w}_{i} !}{{k}_{i}^{1} ! {k}_{i}^{2} ! {k}_{i}^{3} ! {k}_{i}^{d} ! {k}_{i}^{o} !} {P ( {W}_{{α}_{q} β}^{i} )}^{{k}_{i}^{1}} {P ( {W}_{{α}_{r} β}^{i} )}^{{k}_{i}^{2}} {P ( {W}_{{α}_{q} {α}_{r}}^{i} )}^{{k}_{i}^{3}} {P ( {W}_{{α}_{q} {α}_{r} β}^{i} )}^{{k}_{i}^{d}} {P ( {W}_{{α}_{q}}^{i} ⋃ {W}_{{α}_{r}}^{i} ⋃ {W}_{β}^{i} ⋃ {W}_{∅}^{i} )}^{{k}_{i}^{o}} )$$

$$where$$

$$P ( {W}_{{α}_{q} β}^{i} ) = {\sum }_{k = 1}^{{n}_{i}} ( \begin{matrix}{n}_{i} \\ k\end{matrix} ) {{f}_{q}^{k} ( 1 - {f}_{q} - {f}_{r} )}^{{n}_{i} - k} {( 1 - {ε}^{k} )}^{2} + {\sum }_{{n}_{1} = 1}^{{n}_{i} - 1} {\sum }_{{n}_{2} = 1}^{{n}_{i} - {n}_{1}} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} {( 1 - {ε}^{{n}_{1}} )}^{2} {ε}^{{n}_{2}} + {\sum }_{{n}_{1} = 1}^{{n}_{i} - 1} {\sum }_{{n}_{2} = 1}^{{n}_{i} - {n}_{1}} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} ( 1 - {ε}^{{n}_{1}} ) {ε}^{{n}_{1}} ( 1 - {ε}^{{n}_{2}} ) {ε}^{{n}_{2}}$$

$$P ( {W}_{{α}_{r} β}^{i} ) = {\sum }_{k = 1}^{{n}_{i}} ( \begin{matrix}{n}_{i} \\ k\end{matrix} ) {{f}_{r}^{k} ( 1 - {f}_{q} - {f}_{r} )}^{{n}_{i} - k} {( 1 - {ε}^{k} )}^{2} + {\sum }_{{n}_{2} = 1}^{{n}_{i} - 1} {\sum }_{{n}_{1} = 1}^{{n}_{i} - {n}_{1}} \frac{{n}_{i} !}{{n}_{1} ! {n}_{2} ! ( {n}_{i} - {n}_{1} - {n}_{2} ) !} {f}_{r}^{{n}_{1}} {{f}_{q}^{{n}_{2}} ( 1 - {f}_{q} - {f}_{r} )}^{{n}_{i} - {n}_{1} - {n}_{2}} {( 1 - {ε}^{{n}_{2}} )}^{2} {ε}^{{n}_{1}} + {\sum }_{{n}_{2} = 1}^{{n}_{i} - 1} {\sum }_{{n}_{1} = 1}^{{n}_{i} - {n}_{2}} \frac{{n}_{i} !}{{n}_{1} ! {n}_{2} ! ( {n}_{i} - {n}_{1} - {n}_{2} ) !} {f}_{r}^{{n}_{1}} {{f}_{q}^{{n}_{2}} ( 1 - {f}_{q} - {f}_{r} )}^{{n}_{i} - {n}_{1} - {n}_{2}} ( 1 - {ε}^{{n}_{1}} ) {ε}^{{n}_{1}} ( 1 - {ε}^{{n}_{2}} ) {ε}^{{n}_{2}} \\ P ( {W}_{{α}_{q} {α}_{r}}^{i} ) = {\sum }_{{n}_{1} = 1}^{{n}_{i} - 1} {\sum }_{{n}_{2} = 1}^{{n}_{i} - {n}_{1} - 1} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} ( 1 - {ε}^{{n}_{1}} ) {ε}^{{n}_{1}} ( 1 - {ε}^{{n}_{2}} ) {ε}^{{n}_{2}} \\ P ( {W}_{{α}_{q} {α}_{r} β}^{i} ) = {\sum }_{{n}_{1} = 1}^{{n}_{i} - 1} {\sum }_{{n}_{2} = 1}^{{n}_{i} - {n}_{1}} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} {ε}^{{n}_{1}} ( 1 - {ε}^{{n}_{1}} ) {( 1 - {ε}^{{n}_{2}} )}^{2} + {\sum }_{{n}_{1} = 1}^{{n}_{i} - 1} {\sum }_{{n}_{2} = 1}^{{n}_{i} - {n}_{1}} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} {( 1 - {ε}^{{n}_{1}} )}^{2} {( 1 - {ε}^{{n}_{2}} )}^{2} + {\sum }_{{n}_{1} = 1}^{{n}_{i} - 1} {\sum }_{{n}_{2} = 1}^{{n}_{i} - {n}_{1}} \frac{{n}_{i} !}{{n}_{1} ! {n}_{2} ! ( {n}_{i} - {n}_{1} - {n}_{2} ) !} {f}_{q}^{{n}_{1}} {{f}_{r}^{{n}_{2}} ( 1 - {f}_{q} - {f}_{r} )}^{{n}_{i} - {n}_{1} - {n}_{2}} {( 1 - {ε}^{{n}_{1}} )}^{2} {ε}^{{n}_{2}} ( 1 - {ε}^{{n}_{2}} ) \\ P ( {W}_{{α}_{q}}^{i} ⋃ {W}_{{α}_{r}}^{i} ⋃ {W}_{β}^{i} ⋃ {W}_{∅}^{i} ) = 1 - P ( {W}_{{α}_{q} β}^{i} ) - P ( {W}_{{α}_{r} β}^{i} ) - P ( {W}_{{α}_{q} {α}_{r}}^{i} ) - P ( {W}_{{α}_{q} {α}_{r} β}^{i} )$$

The likelihood of observing the data K={kᵢ¹, kᵢ², kᵢ³, kᵢ^{d}, kᵢ^{o}: k=1, 2, . . . , s} under the hypothesis that α_{q}β and αᵣβ represent one dual TCRα clone αᵣα_{q}β is

$$ℒ ( 𝒦 \vert clone {α}_{r} {α}_{q} β with frequency {f}_{d} ) = {\prod }_{i = 1}^{s} ( \frac{{w}_{i} !}{{k}_{i}^{1} ! {k}_{i}^{2} ! {k}_{i}^{3} ! {k}_{i}^{d} ! {k}_{i}^{o} !} {P ( {W}_{{α}_{q} β}^{i} )}^{{k}_{i}^{1}} {P ( {W}_{{α}_{r} β}^{i} )}^{{k}_{i}^{2}} {P ( {W}_{{α}_{q} {α}_{r}}^{i} )}^{{k}_{i}^{3}} {P ( {W}_{{α}_{q} {α}_{r} β}^{i} )}^{{k}_{i}^{d}} {P ( {W}_{{α}_{q}}^{i} ⋃ {W}_{{α}_{r}}^{i} ⋃ {W}_{β}^{i} ⋃ {W}_{∅}^{i} )}^{{k}_{i}^{o}} )$$

$$where$$

$$P ( {W}_{{α}_{q} β}^{i} ) = P ( {W}_{{α}_{r} β}^{i} ) = P ( {W}_{{α}_{q} {α}_{r}}^{i} ) = {\sum }_{k = 1}^{{n}_{i}} ( \begin{matrix}{n}_{i} \\ k\end{matrix} ) {{f}_{d}^{k} ( 1 - {f}_{d} )}^{{n}_{i} - k} {{ε}^{k} ( 1 - {ε}^{k} )}^{2}$$

$$P ( {W}_{{α}_{q} {α}_{r} β}^{i} ) = {\sum }_{k = 1}^{{n}_{i}} ( \begin{matrix}{n}_{i} \\ k\end{matrix} ) {{f}_{d}^{k} ( 1 - {f}_{d} )}^{{n}_{i} - k} {( 1 - {ε}^{k} )}^{3}$$

$$P ( {W}_{{α}_{q}}^{i} ⋃ {W}_{{α}_{r}}^{i} ⋃ {W}_{β}^{i} ⋃ {W}_{∅}^{i} ) = 1 - P ( {W}_{{α}_{q} β}^{i} ) - P ( {W}_{{α}_{r} β}^{i} ) - P ( {W}_{{α}_{q} {α}_{r}}^{i} ) - P ( {W}_{{α}_{q} {α}_{r} β}^{i} )$$

It was assessed empirically that if the difference between the logarithms of the likelihoods derived above is greater than or equal to 10, then chains α_{q}, αᵣ and β should be assumed to comprise a dual TCRα clone.

The above calculation of these full likelihoods is computationally tractable only for wells with less than 50 cells due to the need to calculate large multinomial coefficients. The full-likelihood method is therefore only appropriate for estimating frequencies of those relatively abundant clones that are commonly found in the wells with smaller sample sizes.

Results

This algorithm was tested using simulated data sets reflecting the properties of antigen-specific T cell populations and the experimental pipeline, with sharing of α and β chains, 10% dual TCR clones, and a 15% drop rate of chains due to PCR errors. The algorithm was tested for:

Overall depth: percentage of αβ pairs that are correctly identified.

False pairing rate: percentage of identified αβ pairs that are incorrect.

Depth of top clones: the percentage of the most abundant clones (those comprising 50% of the population) that are correctly identified.

A range of skewed clonal frequency distributions and different sampling strategies were investigated, using either one or five 96-well plates, each with fixed numbers of cells in each well, or varying from 20 to 200 cells/well across the plate. 40 simulations were made per parameter set. The sampling strategies were either 200 cells/well, 300 cells/well or a “mixed” strategy with 1 column of 20 cells/well, 2 of 50 cells/well and 3 each of 100, 200 and 300 cells per well.

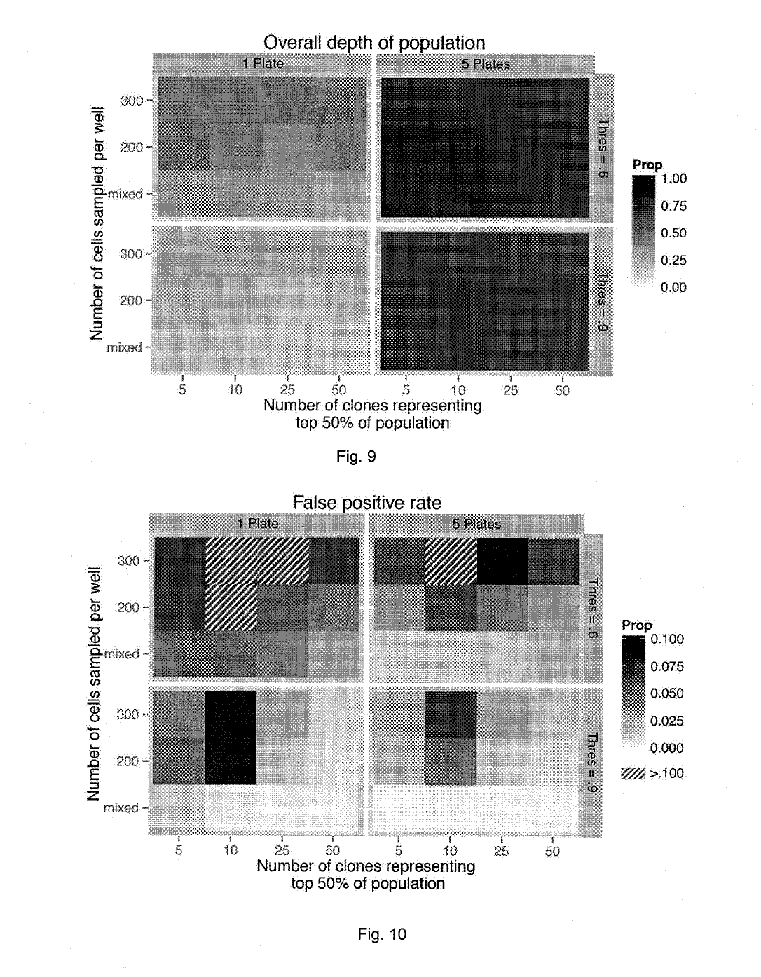

The results are shown in FIGS. 9 to 12. “Thres” in FIGS. 9 to 11 refers to the threshold for acceptance as a “true” pair in step 6 of the method described above.

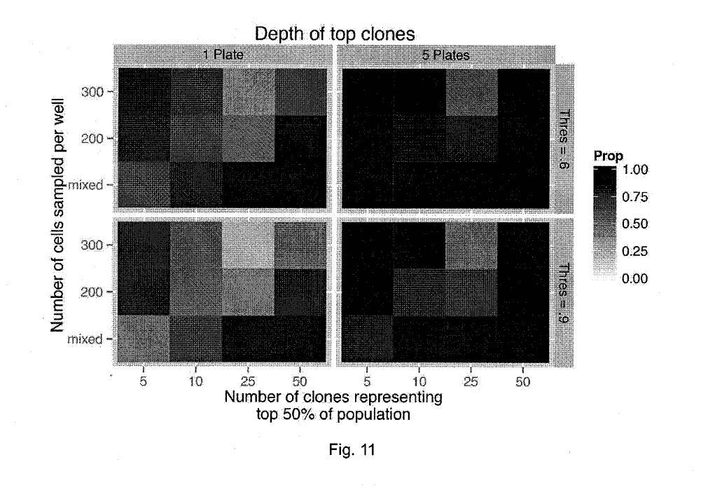

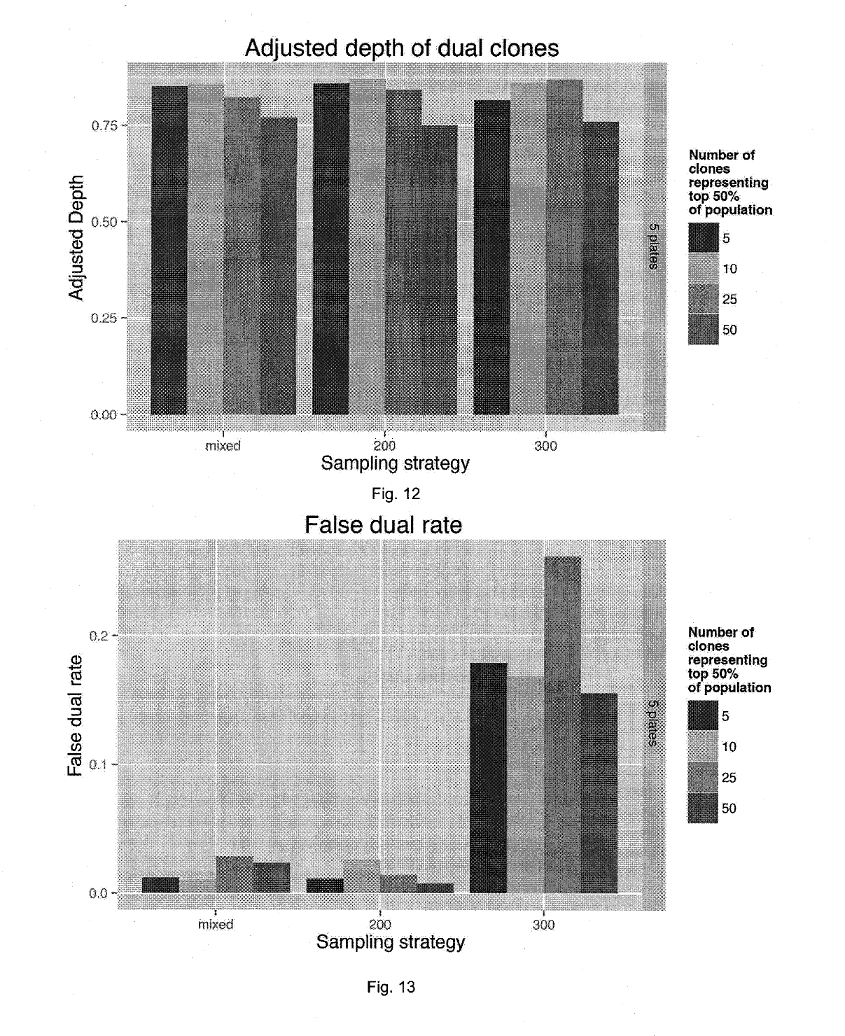

A mixed sampling strategy appears optimal for moderately skewed clonal distributions; ‘top’ clones (those each making up >0.025% of the response) can be recovered from a single plate with FPR <2% (FIG. 10) and depths approaching 100% (FIG. 11). More plates are needed only to obtain substantial increases in overall depth (FIG. 9). Dual TCR are identified at depths of typically 80% (FIG. 12) and distinguished from β chain sharing with greater than 95% accuracy (FIG. 13, which shows “false dual rate”, i.e. the rate at which the algorithm incorrectly assigns two beta sharing as a dual clone)).

Extensive characterization of antigen-specific αβ TCR clones is difficult in part due to the confounding properties of shared chains and dual TCRs antigen-specific populations. The algorithm described addresses these problems and identifies clones with depth and accuracy in an efficient, scalable manner. It may benefit future studies of T cell diversity in the context of vaccine design and autoimmunity.

Further simulations were conducted based on similar principles to those above, simulated data sets reflecting the properties of antigen-specific T cell populations and the experimental pipeline, with sharing of α and β chains, 30% of clones expressing two TCRα, 6% of clones expressing two TCRβ, and the two error rates. For the first error rate (the drop rate), each virtual clone was assigned a drop rate at random from a lognormal distribution with mean 0.15 and standard deviation of 0.01, with the rate capped at 0.9. Each instance of the CDR3α(s) and CDR3β from that clone was then removed from the well with probability equal to the drop rate. For the second error rate, an error rate randomly drawn from a lognormal distribution with mean 0.02 and standard deviation 0.005 was assigned. Each instance of this sequence at the per-cell level was replaced at random by one of three erroneous “daughter” sequences, unique and specific to the parent sequence, with probability equal to this sequence-specific in-frame error rate. Overall, the results were substantially similar to those described above and illustrated in FIGS. 9-13.

The sampling strategies used in these simulations included a high-mixed sampling strategy of 5 plates (480 wells total: 128 wells of 20 cells, 64 wells of 50 cells, 96 wells of 100 cells, 96 wells of 200 cells, and 96 wells of 300 cells), a low-mixed sampling strategy of 5 plates (480 wells total: 96 wells of 15 cells, 32 wells of 20 cells, 64 wells of 30 cells, 96 wells of 50 cells, 96 wells of 100 cells, 96 wells of 150 cells), a constant sampling strategy of 480 wells of 10 cells, and a constant sampling strategy of 480 wells of 25 cells.

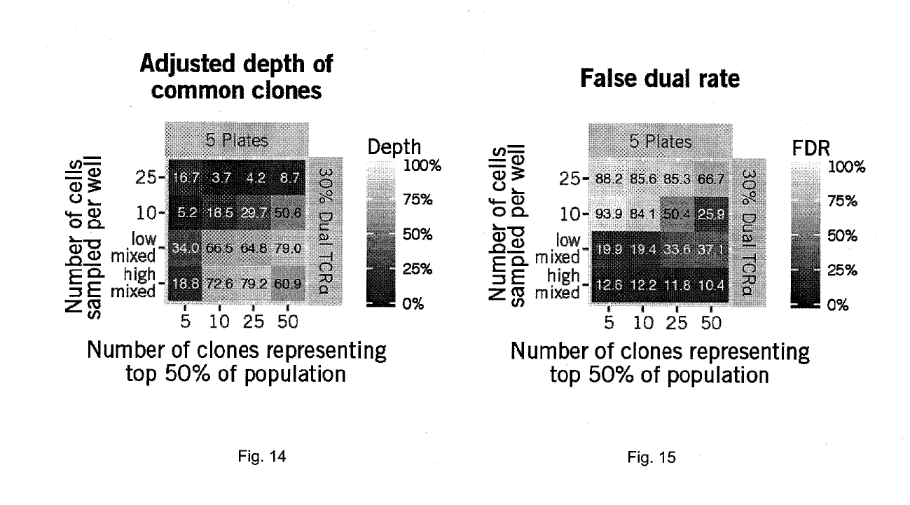

FIGS. 14 and 15 show additional results from these further simulations testing the dual discrimination abilities of the method. Specifically, they show the adjusted top dual depth (FIG. 14): the percentage of the identifiable most abundant dual clones (the clones comprising 50% of the population which express dual TCRα) correctly identified; and (FIG. 15) the false dual rate: percentage of identified dual clones that are incorrect.

Comparison with Single-Cell Sequencing

Experiments were also conducted to determine whether the method of the above embodiments (referred to in the figures as “ALPHABETR”) improves upon single-cell approaches. One way to assess this would have been to take a sample of antigen-specific cells, perform single-cell sequencing on a subset of these cells and apply the above embodiments to the remainder to compare their performance on the same set of parent clones. As an alternative to this, both scenarios were simulated. The advantage of the simulation approach is that it allows (i) triangulation of both methods with the gold-standard of the true sequences, which are not known in practical settings due to dropping of chains and in-frame sequencing errors, and (ii) exploring levels of single-cell sequencing that are currently prohibitively costly.

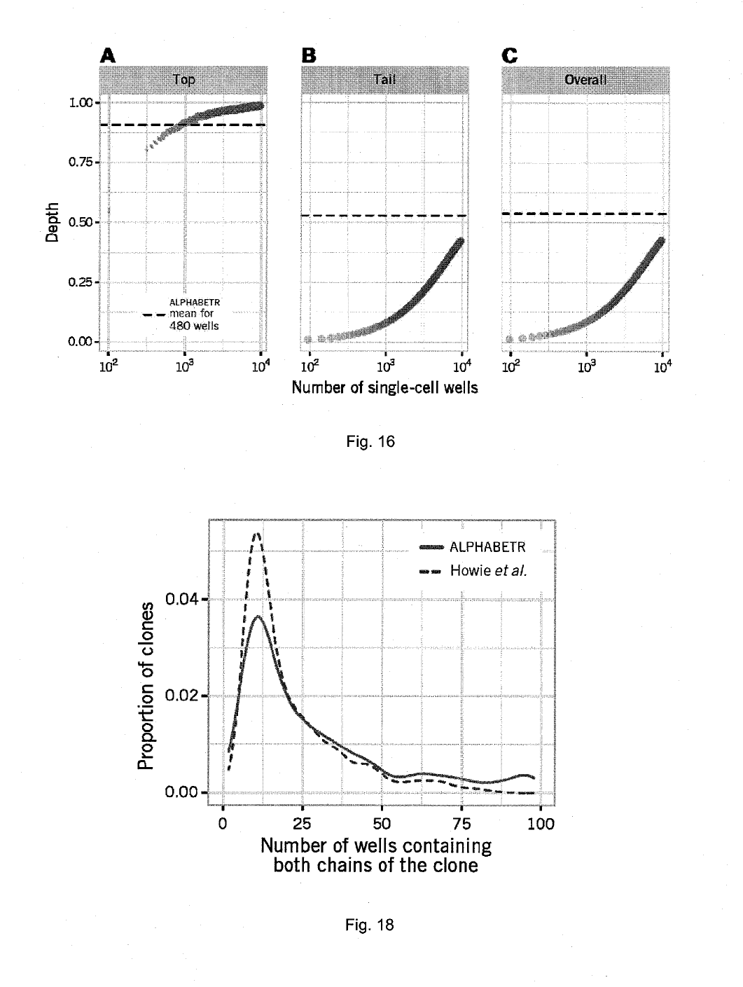

The sequencing of between 96 and 9600 single cells sampled from the same synthetic T cell populations discussed above were simulated using the same model of sequencing errors. FIG. 16 compares the performance of the two methods for a population of 2100 clones, with 25 clones making up the top 50% by abundance. The method of the above embodiments was implemented with the high-mixed sampling strategy of five plates and with a stringency threshold T=0.6. Under the conditions used for FIG. 16, almost double the number of single-cell sequencing runs was required to achieve the same top depth yielded by the above embodiment with five plates, and more than 100 plates of single cells are required to approach the above embodiment's level of recovery of rare clones. With the same clone size distribution, even a single plate analysed with a method according to the above embodiment yields top depths from 78% to 92% depending on the threshold parameter used, whereas 96 single cells yield a top depth of 60%. Single-cell sequencing will exhibit a false positive rate that is approximately twice the mean of the in-frame error rate, or 4% in our simulations, an accuracy that is comparable to that of the above embodiments at their most stringent.

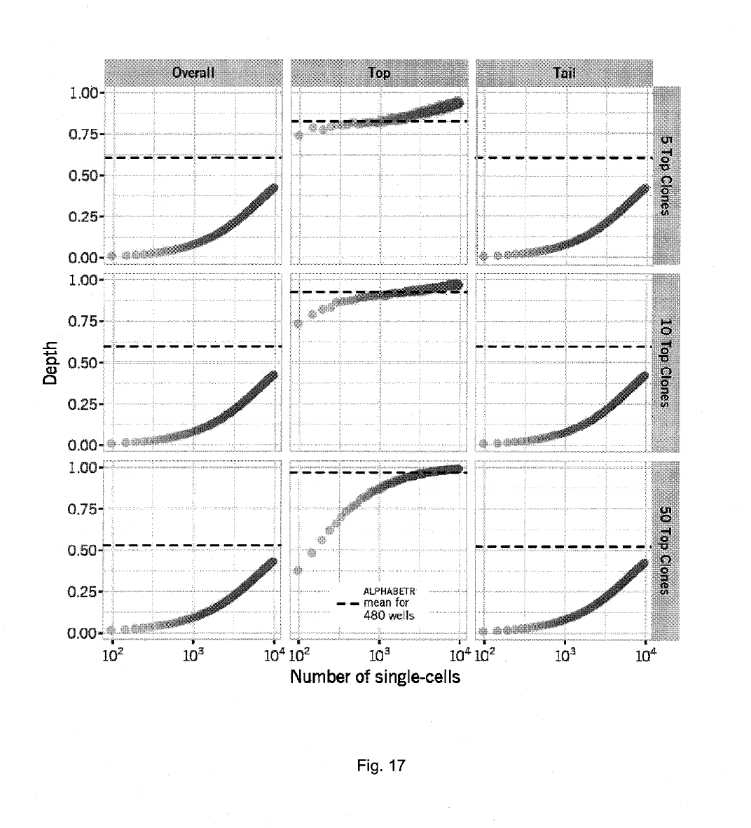

In addition to the comparison discussed above, different skewnesses were explored, that is populations of 2100 clones with 5, 10, and 50 clones comprising the top 50%. The results are shown in FIG. 17 in which the results were evaluated for top depth, tail depth, and overall depth. The dashed lines show the mean performance of the above embodiments with 5 plates using the high-mixed sampling strategy and a threshold of 0.6. The single-cell sequencing results are averages of 200 simulations.

All three sets of additional simulations reinforce the conclusion that substantially larger numbers of samples need to be sequenced with single-cell methods to match the depths achieved by the above embodiments, particularly for rare clones.

Applicability to Real Sequencing Data

Using simulated data allowed the performance of the above embodiments to be assessed directly using the gold standard of known TCRαβ pairs and under a range of plausible experimental conditions. However, to illustrate a real-world application, the above embodiments were also applied to a published dataset derived from the TCRs of tumour-infiltrating lymphocytes (TILs) from human subjects [7]. The study also used a frequency-based method to pair the TCRα and TCRβ obtained by sampling TILs from nine different tumours into the wells of one 96-well plate and sequencing the CDR3α and CDR3β chains found in each. One tumour (Breast 1) yielded only 7 pairs, and was excluded from the analysis.

The above embodiments were applied to the chains from each of the remaining 8 tumours in turn. The pairs determined by the above embodiments were then compared to those identified explicitly in [7] (Table 1 below).

The TIL sequencing data described by Howie et al. [7] provided an opportunity to test the above embodiments on a real TCR sequencing dataset of restricted diversity. The data were obtained by sampling T cells from nine different tumour samples into one 96-well plate and determining the CDR3α and CDR3β sequences in each well. In addition, bulk sequencing of the CDR3α and CDR3β sequences was performed on PMBCs from each patient. This allowed for the association of a subset of the sequences found in the mixed 96-well plate with their tumour sources.

Because the mixed plate contained 561452 unique CDR3α nucleotide sequences and 955987 unique CDR3β nucleotide sequences, these data are too diverse for direct input into the above embodiments. Tumour-specific virtual plates were therefore created by matching CDR3 regions in the wells to the libraries of CDR3 sequences obtained from bulk sequencing of PMBC sampled from each patient. This was done by exact matching of the first 76 bases of the nucleotide sequences of CDR3α pairSEQ sequences and the last 76 bases of the nucleotide sequences of the CDR3α libraries of each tumour sample. For the CDR3β, matching regions were 81 bases in length. These choices reflect the different reads utilised by pairSEQ and immunoSEQ sequencing. Each plate of tumour-specific chains was then analysed using the above embodiments to obtain αβ pairs.

The pairs from the above embodiments were then compared to those pairs determined in [7] for which both the CDR3α and CDR3β chains were explicitly associated with exactly one tumour. Howie et al. also reported clones for which only one chain was associated with a tumour and the other was not found in any of the samples. Since the above embodiments were only applied to the chains from the mixed plate that were definitively associated with one tumour, we removed the partially-matched clones called in [7] from the comparative analysis.

The true TCR clonotypes are unknown and so the aim was to measure degrees of concordance and conflict between the two methods. In 6 out of 8 tumours, the above embodiments recovered fewer clones; however average concordance rates of 77% were found (defined as the proportion of the pairs identified by the above embodiments that were also identified in [7]). Perhaps more strikingly, a very low incidence of conflicting pairs was found (mean 2% across tumours, as a proportion of all pairs identified by the above embodiments). Conflicts were defined as those determined by the two methods that have only one chain in common.

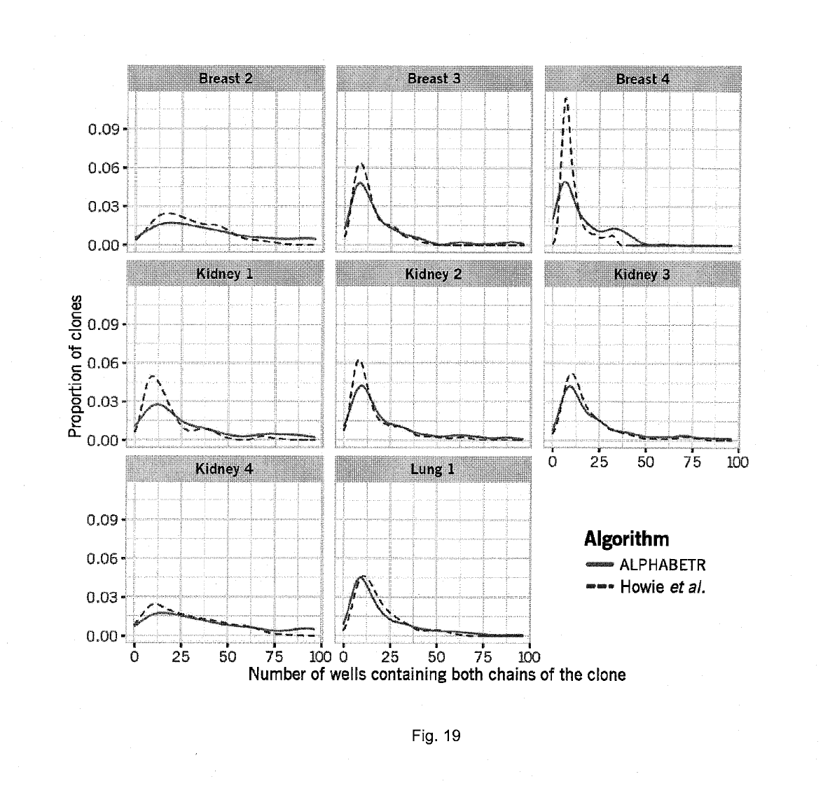

To compare the abilities of the two algorithms to identify rare or common clones, the identified αβ chain pairs were stratified by the frequency with which they co-appeared in wells. Wth stringency thresholds greater than 0.7, it was found that with a single 96-well plate and a sampling strategy optimised for use by the algorithm described in [7], the above embodiments are less efficient at identifying rare clones but identify clones with moderate to high abundances—for which the TCRα and TCRβ chains co-appear in more than a quarter of the wells—more efficiently (see FIG. 18, and FIG. 19 for a breakdown by tumour). The clones identified only by the above embodiments exhibit moderate levels of sharing (TCRα-sharing, mean 16%, range 0-60%; TCRβ-sharing, mean 13%, range 4-31%). Of the sharers, an average of 76% share a chain with a clone that was identified by both methods.

TABLE 1

| | | | | Percentage of | | |
|---|---|---|---|---|---|---|
| | | | | pairs identified | | Number of |
| | Number of pairs | Number of pairs | Number of | by the above | | novel pairs |
| | identified | unambiguously | identical | embodiments | Number of | from the |
| Tumour | by the above | identified by | identified | agreeing with | conflicting | above |
| Sample | embodiments | ref. [7] | pairs | ref. [7] | pairs | embodiments |
| Breast 2 | 98 | 85 | 74 | 75.5% | 0 | 24 |
| Breast 3 | 109 | 129 | 94 | 86.2% | 1 | 14 |
| Breast 4 | 50 | 85 | 26 | 52.0% | 1 | 23 |
| Kidney 1 | 74 | 112 | 58 | 78.4% | 3 | 13 |
| Kidney 2 | 145 | 286 | 126 | 86.9% | 5 | 5 |
| Kidney 3 | 213 | 282 | 166 | 77.9% | 8 | 39 |
| Kidney 4 | 157 | 176 | 131 | 83.4% | 1 | 25 |
| Lung 1 | 173 | 163 | 124 | 71.7% | 1 | 48 |

General Provisions

The systems and methods of the above described embodiments may be implemented in whole or in part in a computer system (in particular in computer hardware or in computer software) in addition to the structural components and interactions described.

The term “computer system” includes the hardware, software and data storage devices for embodying a system or carrying out a method according to the above described embodiments. For example, a computer system may comprise a central processing unit (CPU), input means, output means and data storage. Preferably the computer system has a monitor to provide a visual output display. The data storage may comprise RAM, disk drives or other computer readable media. The computer system may include a plurality of computing devices connected by a network and able to communicate with each other over that network.

The methods of the above embodiments may be provided as computer programs or as computer program products or computer readable media carrying a computer program which is arranged, when run on a computer, to perform the method(s) described above, in particular by controlling the various components of the production line and the temperature measurement apparatus.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

### REFERENCES

[1] DeWitt W S, Emerson R O, Lindau P, et al. (2015). Dynamics of the cytotoxic T cell response to a model of acute viral infection. J Virol 89(8):4517-26

[2] Emerson R O, Sherwood A M, Rieder M J, et al. (2013). High-throughput sequencing of T-cell receptors reveals a homogeneous repertoire of tumour-infiltrating lymphocytes in ovarian cancer. J Pathol 231(4):433-40

[3] Cukalac T, Kan W T, Dash P, et al. (2015). Paired TCR αβ analysis of virus-specific CD8(+) T cells exposes diversity in a previously defined ‘narrow’ repertoire. Immunol Cell Biol 93(9):804-14

[4] Dash P, McClaren J L, Oguin T H 3rd, et al. (2011). Paired analysis of TCRα and TCRβ chains at the single-cell level in mice. J Clin Invest 121(1):288-95

[5] Meijer P J, Andersen P S, Haahr Hansen M, et al. (2006). Isolation of human antibody repertoires with preservation of the natural heavy and light chain pairing. J Mol Biol 358(3):764-72

[6] Reddy S T, Ge X, Miklos A E, et al. (2010). Monoclonal antibodies isolated without screening by analyzing the variablegene repertoire of plasma cells. Nat Biotechnol 28(9):965-9

[7] Howie B, Sherwood A M, Berkebile A D, et al. (2015). High-throughput pairing of T cell receptor and sequences. Sci Transl Med 7(301):301ra131

All references referred to above are hereby incorporated by reference.

1. A method of estimating the clonal composition of a T cell population, the method including the steps of:
obtaining a plurality of samples from said population, each sample containing a plurality of T cells;
sequencing the CDR3α and CDR3β regions of each of those samples;
calculating, for every α and β chain found in common in at least one of the samples, an association score which reflects the likelihood of pairing between each of those chains;
repeatedly, for a randomly selected subset of said samples, determining, from the calculated association scores, the most likely αβ pairs in each of said selected samples and selecting those pairs as candidate αβ pairs;
comparing the number of instances that each of said candidate αβ pairs appears in all of said repeats to a threshold and selecting those pairs whose instances exceed said threshold as true αβ pairs;
estimating, from the frequency of co-existence of α and β chains in each of said samples and the sample size of said sample, the frequency of each of said true αβ pairs; and
discriminating, amongst said αβ pairs, between possible dual T cell receptors or two clones which share a common β chain.

2. The method according to claim 1, wherein the association score for a particular combination of α and β chains is calculated as a function that increases with the number of concurrent appearances of said combination in the samples, weighted inversely by the number of unique α and β chains found in each of the samples in which the combination is present.

3. The method according to claim 1, wherein the random selection is of between 70% and 80% of said samples.

4. The method according to claim 1, wherein the repeated steps are performed at least 100 times.

5. The method according to claim 1, wherein the step of estimating the frequency uses a maximum likelihood approach.

6. The method according to claim 1, wherein the step of discriminating includes determining the ratio of the determined number of co-occurrences of the possible clones in the samples to the number expected if all pairs were distinct clones.

7. The method according to claim 6, wherein the step of discriminating further uses k-means clustering to partition the pairings.

8. The method according to claim 1, wherein the step of discriminating includes determining the likelihoods of all occurrences of dual clones and β-sharing clones under the alternative hypotheses of the clones being dual clones or being β-sharing clones and comparing the differences in the likelihoods obtained.

9. The method according to claim 1, wherein the T cell population is antigen-specific.

10. A method of estimating the clonal composition of a T cell population, the method including the steps of:
obtaining a plurality of samples from said population, each sample containing a plurality of T cells;
sequencing the CDR3α and CDR3β regions of each of those samples;
repeatedly, for a randomly selected subset of said samples:
calculating, for every α and β chain found in common in at least one of the samples in the subset, an association score which reflects the likelihood of pairing between each of those chains; and
determining, from the calculated association scores, the most likely αβ pairs in each of said selected samples and selecting those pairs as candidate αβ pairs;
comparing the number of instances that each of said candidate αβ pairs appears in all of said repeats to a threshold and selecting those pairs whose instances exceed said threshold as true αβ pairs;
estimating, from the frequency of co-existence of α and β chains in each of said samples and the sample size of said sample, the frequency of each of said true αβ pairs; and
discriminating, amongst said αβ pairs, between possible dual T cell receptors or two clones which share a common β chain.

11. The method according to claim 10, wherein the association score for a particular combination of α and β chains is calculated as a function that increases with the number of concurrent appearances of said combination in the samples, weighted inversely by the number of unique α and β chains found in each of the samples in which the combination is present.

12. The method according to claim 10, wherein the random selection is of between 70% and 80% of said samples.

13. The method according to claim 10, wherein the repeated steps are performed at least 100 times.

14. The method according to claim 10, wherein the step of estimating the frequency uses a maximum likelihood approach.

15. The method according to claim 10, wherein the step of discriminating includes determining the ratio of the determined number of co-occurrences of the possible clones in the samples to the number expected if all pairs were distinct clones.

16. The method according to claim 15, wherein the step of discriminating further uses k-means clustering to partition the pairings.

17. The method according to claim 10, wherein the step of discriminating includes determining the likelihoods of all occurrences of dual clones and β-sharing clones under the alternative hypotheses of the clones being dual clones or being β-sharing clones and comparing the differences in the likelihoods obtained.

18. The method according to claim 10, wherein the T cell population is antigen-specific.

19. A system for estimating the clonal composition of a T cell population, the system having:
a plurality of sample holders for receiving a plurality of samples from said population, each sample containing a plurality of T cells;
a sequencer for sequencing the samples; and
a processor, wherein:
the sequencer sequences the CDR3α and CDR3β regions of each of the samples;
the processor is arranged to:
calculate, for every α and β chain found in common in at least one of the samples, an association score which reflects the likelihood of pairing between each of those chains; and
repeatedly, for a randomly selected subset of said samples, determine, from the calculated association scores, the most likely αβ pairs in each of said samples and selecting those pairs as candidate αβ pairs;
compare the number of instances that each of said candidate αβ pairs appears in all of said repeats to a threshold and selecting those pairs whose instances exceed said threshold as true αβ pairs;
estimate, from the frequency of co-existence of α and β chains in each of said samples and the sample sizes, the frequency of each of said true αβ pairs; and
discriminate, amongst said αβ pairs, between possible dual T cell receptors or two clones which share a common β chain.

20. The system according to claim 19, wherein the association score for a particular combination of α and β chains is calculated as a function that increases with the number of concurrent appearances of said combination in the samples, weighted inversely by the number of unique α and β chains found in each of the samples in which the combination is present.

21. The system according to claim 19, wherein the random selection is of between 70% and 80% of said samples.

22. The system according to claim 19, wherein the processor performs the repeated steps at least 100 times.

23. The system according to claim 19, wherein the processor estimates the frequency using a maximum likelihood approach.

24. The system according to claim 19, wherein the processor is arranged to discriminate by determining the ratio of the determined number of co-occurrences of the possible clones in the samples to the number expected if all pairs were distinct clones.

25. The system according to claim 24, wherein the processor further uses k-means clustering to partition the pairings.

26. The system according to claim 19, wherein the processor is arranged to discriminate by determining the likelihoods of all occurrences of dual clones and β-sharing clones under the alternative hypotheses of the clones being dual clones or being β-sharing clones and comparing the differences in the likelihoods obtained.

27. The system according to any one of claim 19, wherein the T cell population is antigen-specific.

28. A system for estimating the clonal composition of a T cell population, the system having:
a plurality of sample holders each for receiving a plurality of samples from said population, each sample containing a plurality of T cells;
a sequencer for sequencing the samples; and
a processor, wherein:
the sequencer is operable to sequence the CDR3α and CDR3β regions of each of the samples;
the processor is arranged to:
repeatedly, for a randomly selected subset of said samples:
calculate, for every α and β chain found in common in at least one of the samples in the subset, an association score which reflects the likelihood of pairing between each of those chains; and
determine, from the calculated association scores, the most likely αβ pairs in each of said samples and selecting those pairs as candidate αβ pairs;
compare the number of instances that each of said candidate αβ pairs appears in all of said repeats to a threshold and selecting those pairs whose instances exceed said threshold as true αβ pairs;
estimate, from the frequency of co-existence of α and β chains in each of said samples and the sample sizes, the frequency of each of said true αβ pairs; and
discriminate, amongst said αβ pairs, between possible dual T cell receptors or two clones which share a common β chain.

29. The system according to claim 28, wherein the association score for a particular combination of α and β chains is calculated as a function that increases with the number of concurrent appearances of said combination in the samples, weighted inversely by the number of unique α and β chains found in each of the samples in which the combination is present.

30. The system according to claim 28, wherein the random selection is of between 70% and 80% of said samples.

31. The system according to claim 28, wherein the processor performs the repeated steps at least 100 times.

32. The system according to claim 28, wherein the processor estimates the frequency using a maximum likelihood approach.

33. The system according to claim 28, wherein the processor is arranged to discriminate by determining the ratio of the determined number of co-occurrences of the possible clones in the samples to the number expected if all pairs were distinct clones.

34. The system according to claim 33, wherein the processor further uses k-means clustering to partition the pairings.

35. The system according to claim 28, wherein the processor is arranged to discriminate by determining the likelihoods of all occurrences of dual clones and β-sharing clones under the alternative hypotheses of the clones being dual clones or being β-sharing clones and comparing the differences in the likelihoods obtained.

36. The system according to claim 28, wherein the T cell population is antigen-specific.
